(19) United States
(12) Patent Application Publication
(10) Pub. No.: US 2007/0038031 A1
(43) Pub. Date: Feb. 15, 2007

(54) Endoscope distal end part

(75) Inventors: Masaaki Miyagi, Tokyo (JP); Seisuke Takase, Tokyo (JP); Hidenobu Kimura, Tokyo (JP)

Correspondence Address: Thomas Spinelli;Scully, Scott, Murphy & Presser, 400 Garden City Plaza, Garden City, NY, 11530, US

(73) Assignee: Olympus Medical Systems Corp., Tokyo (JP)

(21) Appl. No.: 11/200,400

(22) Filed: Aug. 9, 2005

(51) Int. Cl. A61B 1/06; A61B 1/12; A61B 1/00

(52) U.S. Cl. 600/182; 600/178; 600/156; 600/129

(57) ABSTRACT

An endoscope distal end part of the invention includes a first illumination hole in which a first light guide is to be placed, a second illumination hole in which a second light guide is to be placed, a third illumination hole in which a third light guide is to be placed, and an observation hole which is at least partially positioned in an area surrounded by each of the illumination holes and in which an image pickup unit is to be placed.

### BACKGROUND OF THE INVENTION

1. Field of the Invention

The present invention relates to an endoscope distal end part provided at the distal end of an insertion portion of an endoscope including an observation optical system having a wide angular field of view.

2. Description of the Related Art

As well known, an endoscope is widely used in the medical field and so on. An endoscope has a long and narrow insertion portion to be inserted into a body cavity, and can observe an organ in the body cavity or perform some treatment by using a treatment device being inserted into an insertion channel of the treatment device as required.

The distal end of the insertion portion has a bending section and a rigid endoscope distal end part (called distal rigid part, hereinafter). An operation portion of the endoscope is operated to bend the bending section so that the direction of observation through an objective lens of the observation optical system provided in an image pickup unit placing hole in the distal rigid part can be changed.

For wider range of observation, various endoscopes have been proposed each of which has an observation optical system having a wider angular field of view. For example, Japanese Unexamined Patent Application Publication No. 2001-258823 discloses an endoscope including an observation optical system having a wider, around 140 to 210°, angular field of view of observation in the image pickup unit placing hole of the distal rigid part.

In the endoscope disclosed in Japanese Unexamined Patent Application Publication No. 2001-258823 with the observation optical system having a wider angular field of view, an optical axis of the distal side of a light guide fiber bundle for illuminating inside of a body cavity is disposed in a light guide placing through hole within the distal rigid part with the optical axis tilted outward at a predetermined angle with respect to the optical axis of the observation optical system in order to uniformly irradiate light to the entire observation part in the body cavity.

### SUMMARY OF THE INVENTION

Briefly, an endoscope distal part of the invention includes a first light guide placing through hole in which a first light guide is placed, a second light guide placing through hole in which a second light guide is placed, a third light guide placing through hole in which a third light guide is placed, and an image pickup unit placing hole which is at least partially positioned in an area surrounded by each of the light guide placing through holes and in which an image pickup unit is placed.

The above and other objects, features and advantages of the invention will become more clearly understood from the following description referring to the accompanying drawings.

### BRIEF DESCRIPTION OF THE DRAWINGS

FIG. 1 is a front view schematically showing a configuration of an endoscope apparatus including an endoscope distal part according to an embodiment of the invention;

FIG. 2 is a front view of the distal surface of an insertion portion of the endoscope;

FIG. 3 is a sectional view schematically showing a structure of the distal part taken along the Line III-III in FIG. 2;

FIG. 4 is a sectional diagram schematically showing a structure of the distal part taken along the Line IV-IV in FIG. 2;

FIG. 5 is a front view of the distal rigid part in FIGS. 3 and 4 from the distal side;

FIG. 6 is a perspective view including a partial sectional view taken along the Line VI-VI in FIG. 5;

FIG. 7 is a perspective view including a partial sectional view taken along the Line VII-VII in FIG. 5;

FIG. 8 is a back view of the distal rigid part in FIGS. 3 and 4 from the proximal side;

FIG. 9 is a side view of the distal rigid part in FIGS. 3 and 4 from one side; and

FIG. 10 is an enlarged sectional view showing only the distal rigid part and distal cap in FIG. 4.

### DESCRIPTION OF THE PREFERRED EMBODIMENTS

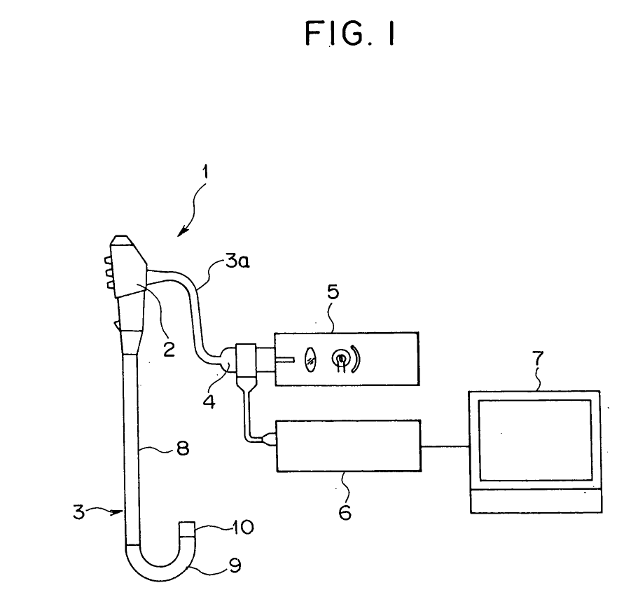

An embodiment of the invention will be described below with reference to drawings. FIG. 1 is a front view schematically showing a configuration of an endoscope apparatus including an endoscope distal part according to an embodiment of the invention.

As shown in FIG. 1, the endoscope apparatus includes a wide-angle endoscope (called “endoscope” hereinafter) 1, a light source device 5, a processor 6 and a monitor 7. The endoscope 1 includes an operation portion 2, an insertion portion 3 and a universal cord 3a. The operation portion 2 is for bending operation and control of several conduits. The insertion portion 3 is connected to the operation portion 2 on the proximal side and is inserted into a body cavity. The universal cord 3a extends from the operation portion 2 and has a connector portion 4 at the distal end. The light source device 5 and the processor 6 are connected to the connector portion 4, and the monitor 7 is connected to the processor 6.

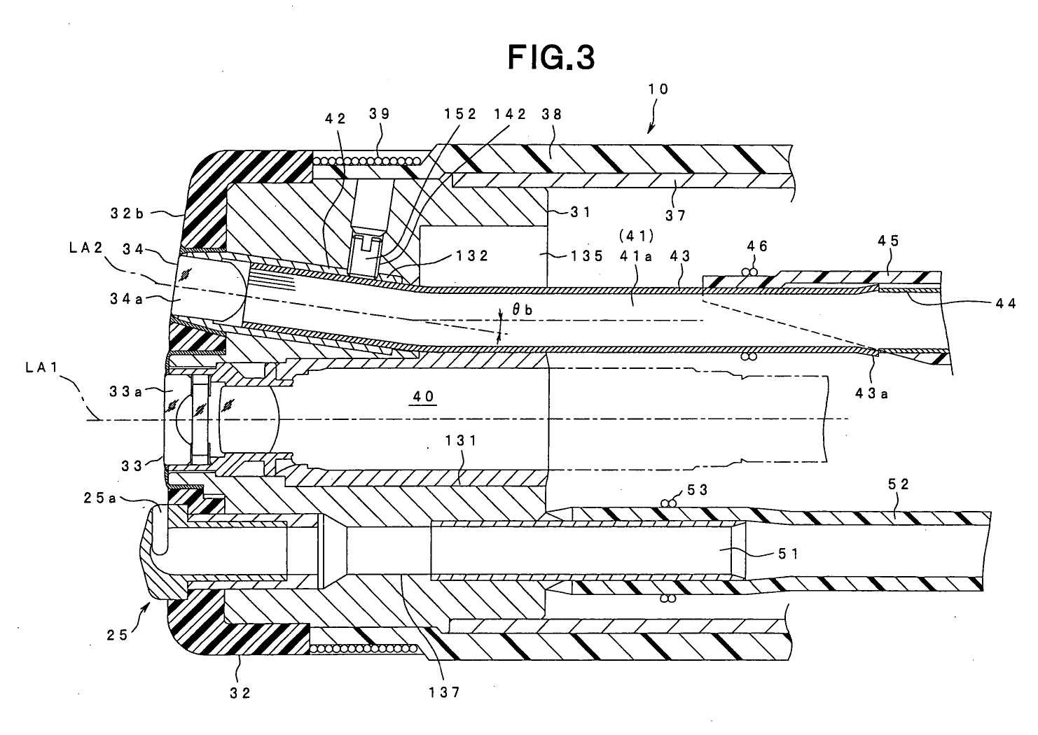

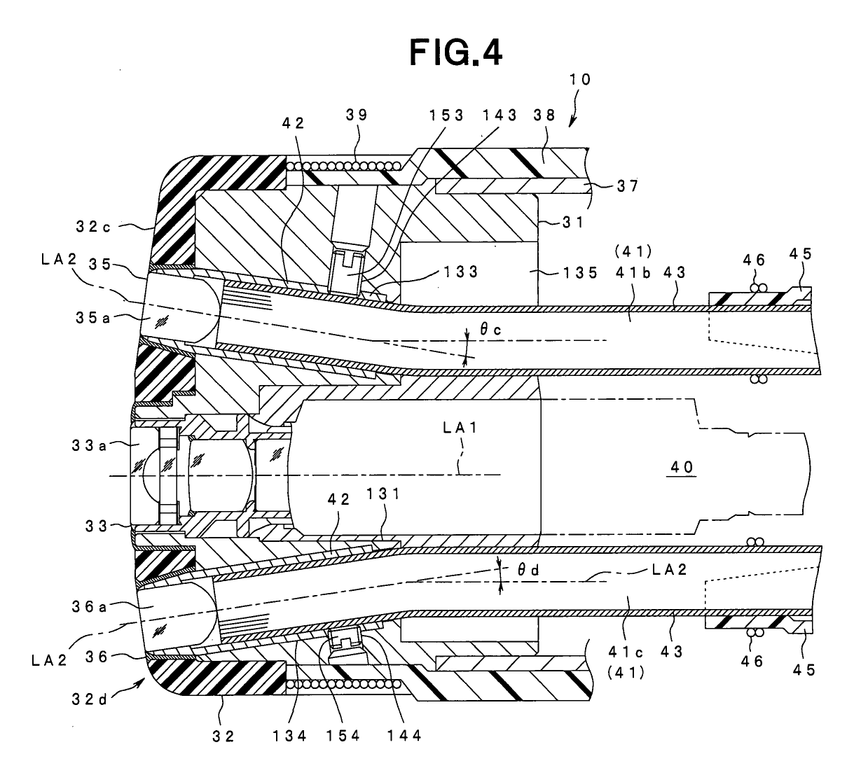

The insertion portion 3 of the endoscope 1 has a flexible tube section 8 having flexibility, a bending section 9 provided on the distal side of the flexible tube section 8, and a distal end section 10 provided on the distal side of the bending section 9. The distal surface of the distal end section 10 has a bullet form, and the distal end section 10 internally includes an image pickup unit 40 (see FIGS. 3 and 4).

The operation portion 2 has an operation knob that remotely bends the bending section 9 and a press button switch for an operation such as air feeding and water feeding.

The light source device 5 has an illumination light source and is connected to a light guide (not shown), which will be described later, through the connector portion 4. The light guide extends through the universal cord 3a, operation portion 2 and insertion portion 3. Furthermore, according to this embodiment, the light source device 5 internally includes an air/water-feeding/sucking source for feeding and/or sucking air and/or water to/from the conduit in the endoscope 1.

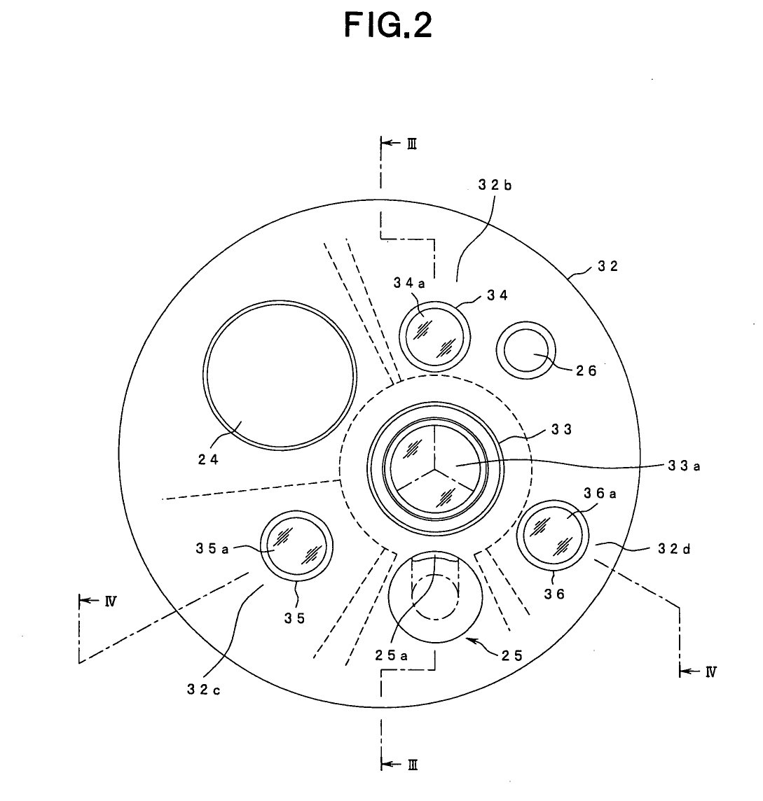

FIG. 2 is a front view of the distal surface of the insertion portion of the endoscope in FIG. 1. FIG. 3 is a sectional diagram schematically showing a structure of the distal end part taken along the Line III-III in FIG. 2. FIG. 4 is a sectional diagram schematically showing a structure of the distal part taken along the Line IV-IV in FIG. 2.

As shown in FIGS. 3 and 4, the distal end section 10 of the endoscope 1 includes a distal rigid part 31 on the distal side. The distal rigid part 31 serves as an endoscope distal end part. The distal end part of a bending distal piece 37 is fixed on the outer periphery at the rear end of the distal rigid part 31. The rear part of the distal rigid part 31 and the bending distal piece 37 are covered by a casing tube 38. The distal end of the casing tube 38 is fixed on the outer periphery of the distal rigid part 31 with threading 39.

A distal cap 32 is attached and fixed to the distal surface of the distal rigid part 31. As shown in FIG. 2, the distal surface of the distal cap 32 has an opening serving as an observation window 33 at a position slightly deviated from the center to one direction and openings serving as first to third illumination windows 34 to 36 around the observation window 33.

An area between the first illumination window 34 and the second illumination window 35 has an opening serving as a suction inlet 24. An area between the second illumination window 35 and the third illumination window 36 has an air/water feeding nozzle 25. An area between the first illumination window 34 and the third illumination window 36 has a front water feeding outlet 26.

An objective lens 33a is provided within the observation window 33. The objective lens 33a has a wide angular field of view and serves as an observation optical system having an angular field of view of about 140 to 210°, for example. The objective lens 33a is for observation of a subject illuminated by illumination light. First to third illumination lenses 34a, 35a and 36a are provided within the first to third illumination windows 34 to 36, respectively.

A tissue of an affected part within the body cavity is sucked through the suction inlet 24. The air/water feeding nozzle 25 is for air/water feeding to the objective lens 33a through an opening 25a in order to clean a stain on the objective lens 33a. Water is fed to the front through the front water feeding outlet 26 in order to clean blood and/or mucus within the body cavity.

The distal rigid part 31 has first to third illumination holes 132 to 134 communicating to the first to third illumination windows 34 to 36 and an observation hole 131 in an area surrounded by the first to third illumination holes 132 to 134, which will be described later in more detail. The first to third illumination holes 132 to 134 serve as first to third light guide placing through holes extending through the observation window 33. The observation hole 131 serves as an image pickup unit placing hole.

The distal rigid part 31 further has a suction inlet hole 136 communicating with the suction inlet 24, an air/water feeding nozzle hole 137 and a front water feeding nozzle 138, which will be described later in more details. The air/water feeding nozzle 25 is attached into the air/water feeding nozzle hole 137. The front water feeding outlet 26 is attached to the front water feeding nozzle hole 138.

The image pickup unit 40 serving as image pickup means extends through the observation hole 131. The image pickup unit 40 is fixed to the observation hole 131 with a fixing screw (not shown) screwed from a side of the distal rigid part 31. The observation hole 131 serves as means for placing an image pickup unit according to this embodiment.

The image pickup unit 40 has a main part including the objective lens 33a and an image pickup device such as a CCD or a CMOS at the back of the objective lens 33a for picking up an image of a subject observed through the objective lens 33a.

The endoscopic image captured by the image pickup device provided in the image pickup unit 40 is photoelectrically converted, transmitted to the processor 6 as an image signal, undergoes predetermined image processing in the processor 6 and then is displayed on the monitor 7 (see FIG. 1).

A frame body 42 is attached to each of the first to third illumination holes 132 to 134, and the distal cap 32 is fixed to the distal end of the frame body 42 with an adhesive. The first to third illumination lenses 34a to 36a are attached and fixed to the frame bodies 42, respectively, with an adhesive. Notably, the first to third illumination holes 132 to 134 serve as first to third means for placing light guides according to this embodiment.

Furthermore, the frame bodies 42 have first to third light guides 41a to 41c at the back of the illumination lenses 34a to 36a such that the distal surfaces of the first to third light guides (LG) 41a, 41b and 41c branching off from one light guide 41 and serving as first to third illumination units can be abutted on the first to third light guides 41a to 41c.

Therefore, the image pickup unit 40 is placed in an area surrounded by the first to third light guides 41a to 41c.

Notably, the distal sides of the first to third light guides 41a to 41c and frame bodies 42 tilt outward by tilting angles of θb to θd, respectively, in the range of 0° to 15° with respect to the image pickup unit 40.

Each of the first to third light guides 41a to 41c includes multiple light guide fiber bundles, which are bundled into one light guide 41 in the middle of the insertion portion 3, and the one bundled light guide 41 is connected to the light source device 5 through the operation portion 2 and the connector portion 4. Thus, the light guide 41 receives illumination light from the illumination light source internally contained in the light source device 5 and transmits the same to the distal side.

The illumination light beams from the first to third light guides 41a to 41c are emitted to the front at the tilting angles θb to θd, respectively, in the range of 0° to 15°, for example, with respect to an insertion axis LA1 of the image pickup unit 40 in the observation hole 131. Notably, the tilting angles cover the range of field of view of the image pickup unit 40, and the tilting angles θb to θd are defined in accordance with the angular field of view of observation of the image pickup unit 40.

A metal pipe 43 is inserted through and held around the periphery of the distal side of each of the first to third light guides 41a to 41c and is covered by a soft protective tube 44 at the rear part.

The distal ends of the metal pipes 43 internally holding the first to third light guides 41a to 41c are attached to the respective frame bodies 42, and the distal ends of the metal pipes 43 are fixed with fixing screws 152, 153 and 154 screwed through the holes 142, 143 and 144, respectively, on the side of the distal rigid part 31.

A rear end part 43a of each of the metal pipes 43 expands and opens in a trumpet form and has the end face abutted on the distal end of the protective tube 44. Thus, the distal end of the protective tube 44 is seated on the rear end part 43a expanded and opened in a trumpet form of each of the metal pipes 43 so that the movement toward the distal end can be limited.

Furthermore, the rear part of each of the metal pipes 43 and the protective tube 44 are covered by a casing tube 45, and the distal part of the casing tube 45 is fixed to the metal pipe 43 with threading 46.

The distal end of the casing tube 45 is obliquely cut so as to allow easy attachment to the metal pipe 43. The distal parts of the first to third light guides 41a to 41c and distal parts of the metal pipes 43 covering the first to third light guides 41a to 41c bend at the same angles as the tilting angles θb to θd of the first to third light guides 41a to 41c and are attached and fixed to the frame bodies 42 with the tilting angles θb to θd kept.

The surface having the openings serving as the illumination windows 34 to 36 of the distal cap 32 has tapered surfaces 32b, 32c and 32d substantially orthogonal to insertion axes LA2 of the light guides.

A connecting tube 51 is attached and fixed from the back into the air/water feeding nozzle hole 137 of the distal rigid part 31 attached to the proximal side of the air/water feeding nozzle 25, and a water feeding tube 52 is connected to the connecting tube 51. Furthermore, the distal part of the water feeding tube 52 is fixed to the connecting tube 51 with threading 53.

A suction channel, not shown, is connected to the suction inlet hole 136 bored through the distal rigid part 31. The suction channel is also used as a treatment device channel. The suction channel is connected to a suction source provided in the light source device 5 through the insertion portion 3, operation portion 2, universal cord 3a and connector portion 4.

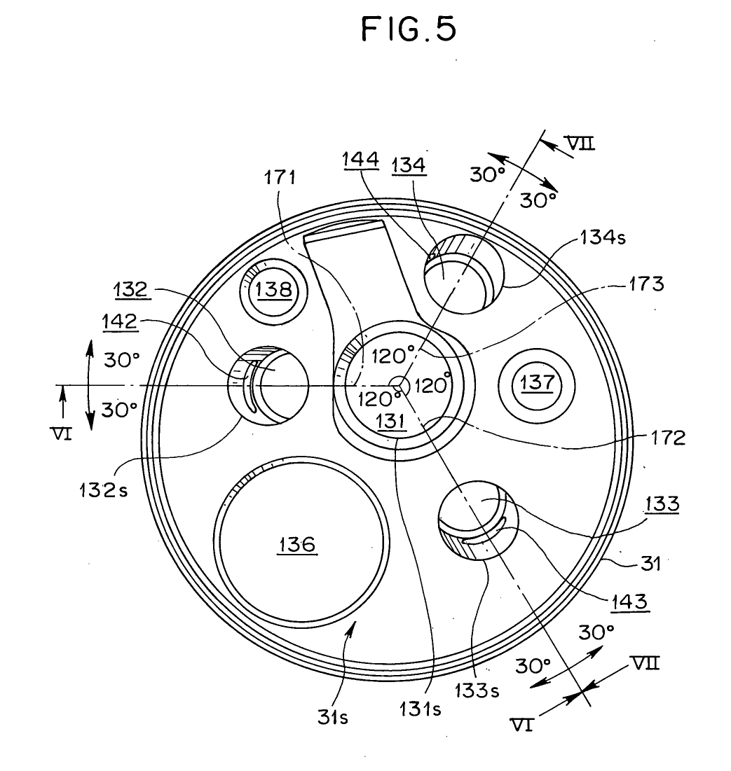

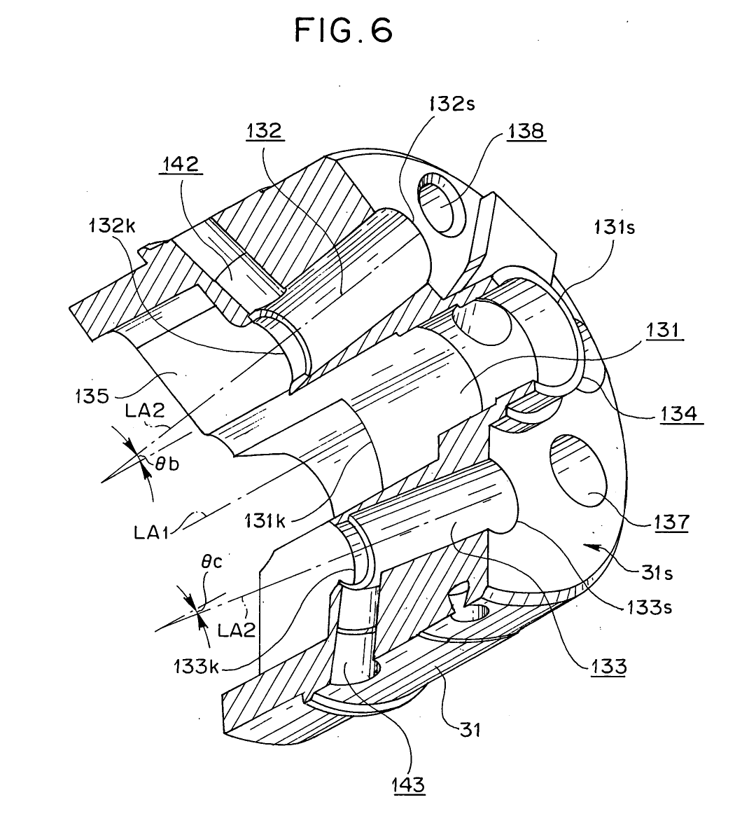

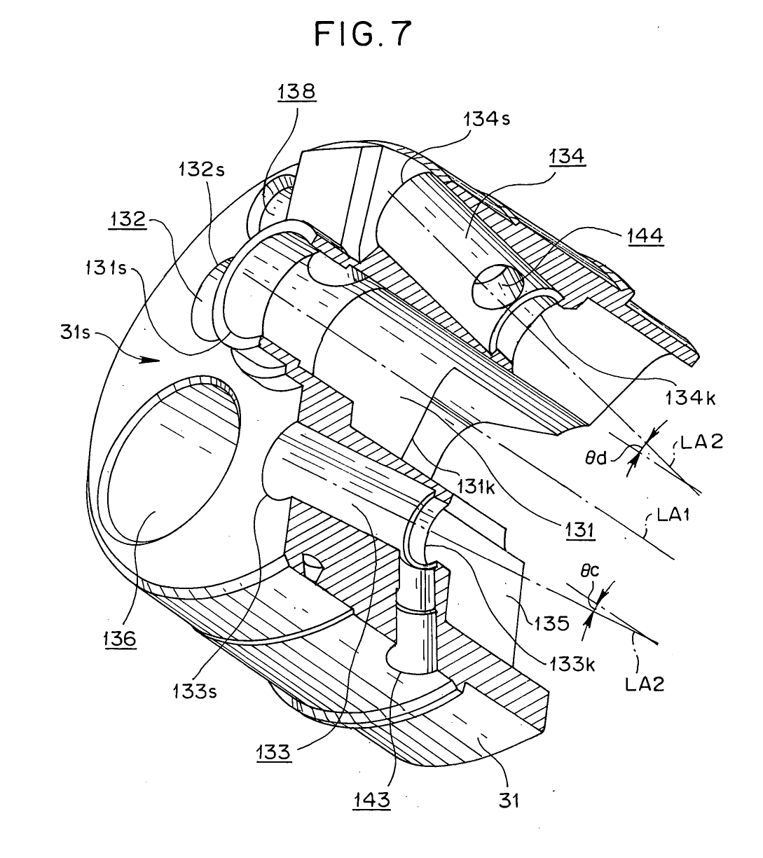

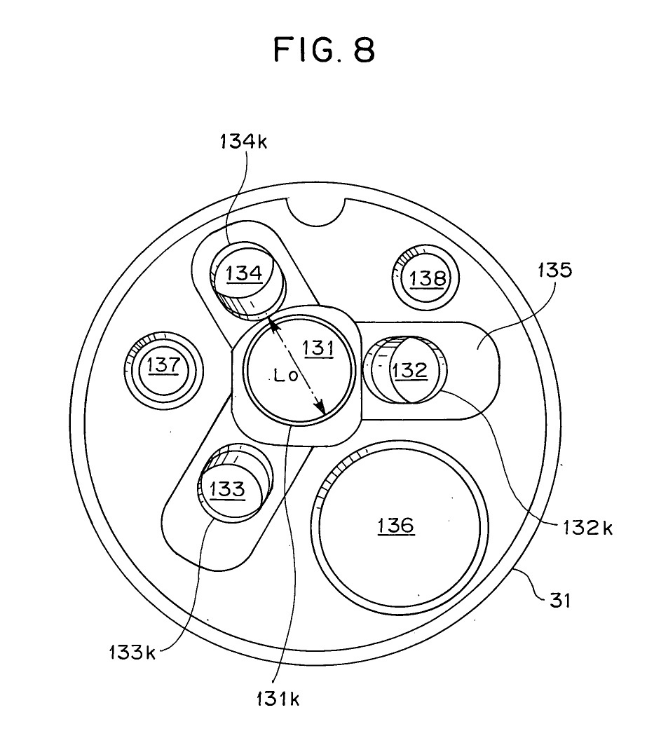

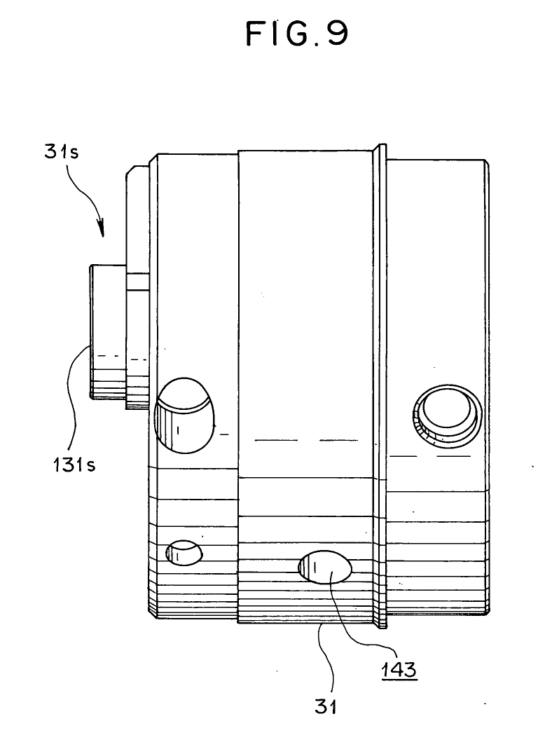

Next, a structure of the distal rigid part 31 will be described with reference to FIGS. 5 to 9. FIG. 5 is a front view of the distal rigid part in FIGS. 3 and 4 from the distal side. FIG. 6 is a perspective view including a partial sectional view taken along the Line VI-VI in FIG. 5. FIG. 7 is a perspective view including a partial sectional view taken along the Line VII-VII in FIG. 5. FIG. 8 is a back view of the distal rigid part in FIGS. 3 and 4 from the proximal side. FIG. 9 is a side view of the distal rigid part in FIGS. 3 and 4 from one side.

As shown in these drawings, the distal rigid part 31 contains metal such as aluminum and stainless and has a cylindrical form having a short axis along the direction of insertion of the endoscope insertion portion 3, for example, and has, for example, three illumination holes through the distal rigid part 31 in the direction of insertion of the distal rigid part 31. Light guides are placed within the illumination holes.

More specifically, the distal rigid part 31 has a first illumination hole 132, a second illumination hole 133 and a third illumination hole 134. The first light guide 41a is placed within the first illumination hole 132. The second light guide 41b is placed within the second illumination hole 133. The third light guide 41c is placed within the third illumination hole 134.

As a result, the distal surface 31s of the distal rigid part 31 has distal openings 132s, 133s and 134s of the first to third illumination holes 132 to 134, and the proximal side of the distal rigid part 31 has proximal openings 132k, 133k and 134k of the first to third illumination holes 132 to 134.

Notably, when the first to third light guides 41a to 41c are placed within the first to third illumination holes 132 to 134, the first to third illumination lenses 34a to 36a are placed within the distal openings 132s to 134s of the first to third illumination holes 132 to 134 through the frame bodies 42.

An area surrounded by three holes of the first illumination hole 132, second illumination hole 133 and third illumination hole 134 at least partially has the observation hole 131 all through the distal rigid part 31 in the direction of insertion of the distal rigid part 31.

As a result, the distal surface 31s of the distal rigid part 31 has a distal opening 131s of the observation hole 131, and the proximal side of the distal rigid part 31 has a proximal opening 131k of the observation hole 131.

Notably, when the image pickup unit 40 is placed in the distal rigid part 31, the objective lens 33a is placed within the distal opening 131s of the observation hole 131. The distal opening 131s of the observation hole 131 is projected from the distal openings 132s to 134s of the first to third illumination holes 132 to 134 at the distal part in the direction of insertion as shown in FIGS. 6 and 7.

Here, the area surrounded by the three holes of the first illumination hole 132, second illumination hole 133 and third illumination hole 134 is within an area defined by a circle formed by connecting edges, which are facing toward each other, of the first to third illumination holes 132 to 134.

Alternatively, the area surrounded by the first to third illumination holes 132 to 134 may be within an area defined by a circle formed by connecting edges, which are not facing, of the first to third illumination holes 132 to 134 or may be within an area formed by connecting the centers of the insertion axes LA2 of the first to third light guides 41a to 41c of the first to third illumination holes 132 to 134 with a line.

The insertion axes LA2 of the first to third illumination holes 132 to 134 tilt outward in the direction of insertion by the tilting angles θb to θd in the range of 0° to 15°, for example, with respect to the insertion axis LA1 of the image pickup unit 40 in the observation hole 131.

In other words, the first to third illumination holes 132 to 134 tilt outward in the direction of insertion by the tilting angles θb to θd in the range of 0° to 15°, for example, with respect to the observation hole 131.

As a result, the openings 132s to 134s on the distal side of the first to third illumination holes 132 to 134 are spaced more apart than the spaces among the openings 132k to 134k on the proximal side of the first to third illumination holes 132 to 134.

More specifically, the centers of the openings 132s to 134s on the distal side of the first to third illumination holes 132 to 134 are spaced apart by 2 to 5 mm from the center of the opening 131s of the distal end of the observation hole 131.

Much more specifically, as shown in FIG. 5, the center of the distal opening 132s of the first illumination hole 132 is positioned on a first line 171 connecting the center of the opening 132s and the center of the distal opening 131s of the observation hole 131 on the distal surface 31s of the distal rigid part 31 such that the angle formed by the first line 171 and a second line 172 connecting the center of the distal opening 133s of the second illumination hole 133 and the center of the distal opening 131s of the observation hole 131 on the distal surface 31s of the distal rigid part 31 can be 90° to 150°, such as 120°.

In other words, when the first illumination lens 34a is placed in the first illumination hole 132, the center of the first illumination lens 34a placed in the distal opening 132s is also positioned on the first line 171.

The center of the distal opening 133s of the second illumination hole 133 is positioned on the second line 172 on the distal surface 31s of the distal rigid part 31. In other words, when the second illumination lens 35a is placed in the second illumination hole 133, the center of the second illumination lens 35a placed in the distal opening 133s is also positioned on the second line 172.

Furthermore, the center of the distal opening 134s of the third illumination hole 134 is positioned on a third line 173 connecting the center of the opening 134s and the center of the distal opening 131s of the observation hole 131 on the distal surface 31s of the distal rigid part 31 such that the angle formed by the third line 173 and at least one of the first line 171 and the second line 172 can be 90° to 150°, such as 120°.

In other words, when the third illumination lens 36a is placed in the third illumination hole 134, the center of the third illumination lens 36a placed in the distal opening 134s is also positioned on the third line 173.

The proximal side of the distal rigid part 31 has a radiant concave 135 enclosing the first to third illumination holes 132 to 134 about the observation hole 131 as shown in FIGS. 6 to 8.

In other words, on the proximal side of the distal rigid part, the observation hole 131 communicates with the first to third illumination holes 132 to 134 without any thickness between the proximal observation hole 131 and the first to third illumination holes 132 to 134 on the proximal side.

In the direction of insertion of the distal rigid part 31, the area between the first illumination hole 132 and the second illumination hole 133 has the suction inlet hole 136 communicating with the suction inlet 24 such that the suction inlet hole 136 can extend through the distal rigid part 31.

Therefore, the area between the first light guide 41a and the second light guide 41b has the suction inlet hole 136 as shown in FIG. 5 when the first and second light guides 41a and 41b are placed in the first and second illumination holes 132 and 133.

In the direction of insertion of the distal rigid part 31, the area between the second illumination hole 133 and the third illumination hole 134 has the air/water feeding nozzle hole 137 to which the air/water feeding nozzle 25 is attached such that the air/water feeding nozzle hole 137 can extend through the distal rigid part 31.

Therefore, the area between the second light guide 41b and the third light guide 41c has the air/water feeding nozzle hole 137 as shown in FIG. 5 when the second and third light guides 41b and 41c are placed in the second and third illumination holes 133 and 134.

In the direction of insertion of the distal rigid part 31, the area between the third illumination hole 134 and the first illumination hole 132 has the front water feeding nozzle hole 138 to which the front water feeding hole 26 is attached such that the front water feeding nozzle hole 138 can extend through the distal rigid part 31.

Therefore, the area between the third light guide 41c and the first light guide 41a has the front water feeding nozzle hole 138 as shown in FIG. 5 when the first and third light guides 41a and 41c are placed in the first and third illumination holes 132 and 134.

Furthermore, the center axes of the suction inlet hole 136, air/water feeding nozzle hole 137 and front water feeding nozzle hole 138 are parallel to the insertion axis LA1.

The side of the distal rigid part 31 has the screw hole 142, as shown in FIG. 6, into which the fixing screw 152 for fixing the metal pipe 43 is screwed. The screw hole 142 communicates with the first illumination hole 132.

Furthermore, the side of the distal rigid part 31 has the screw hole 143, as shown in FIGS. 6, 7 and 9, into which the fixing screw 153 for fixing the metal pipe 43 is screwed. The screw hole 143 communicates with the second illumination hole 133.

Furthermore, the side of the distal rigid part 31 has the screw hole 144, as shown in FIG. 7, into which the fixing screw 154 for fixing the metal pipe 43 is screwed. The screw hole 144 communicates with the third illumination hole 134.

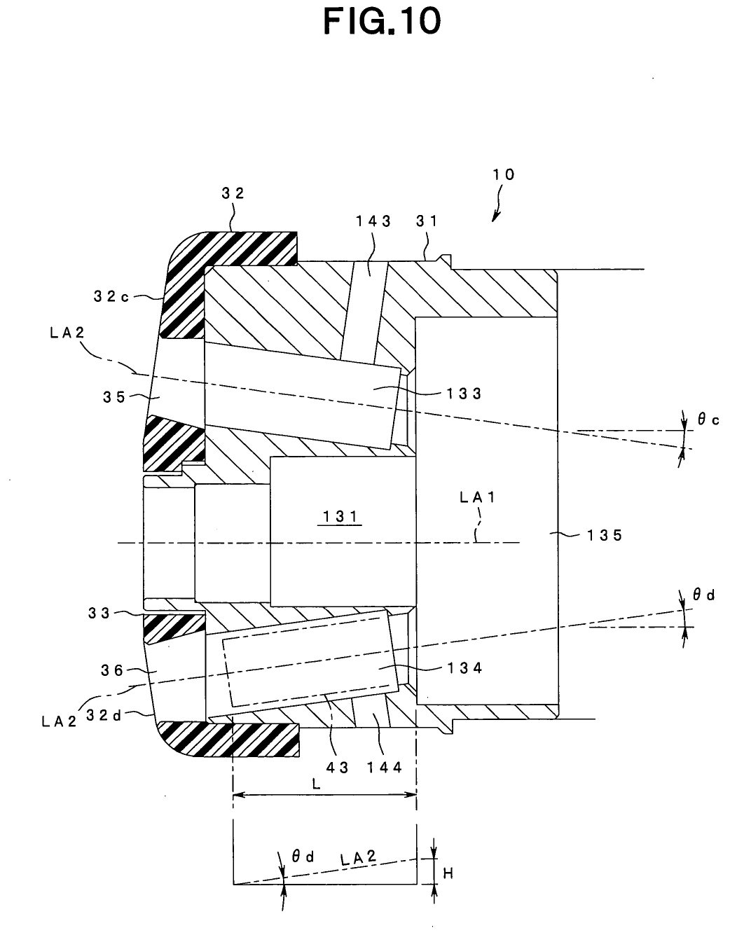

Next, operations of the distal rigid part 31 having the structure and endoscope 1 having the distal rigid part 31 in the distal end section 10 will be described with reference to FIGS. 1 to 9 and 10. FIG. 10 is an enlarged sectional view showing only the distal rigid part and distal cap in FIG. 4.

The illumination light output from the illumination light source contained in the light source device 5 is transmitted toward the distal end of the endoscope through the one light guide 41 extending through the universal cord 3a, operation portion 2 and insertion portion 3 (see FIG. 1).

The one light guide 41 branches off to three light guides 41a to 41c in the middle of the endoscope insertion portion 3, and a subject is illuminated by light emitted from the first to third light guides 41a to 41c to the front of the endoscope 1.

Then, the reflective light from the subject is received and photoelectrically converted by an image pickup device such as a CCD and a CMOS in the image pickup unit 40 and is transmitted to the processor 6 as an image signal. The processor 6 performs predetermined image processing on the received image signal and displays an observation image on the monitor 7 (see FIG. 1).

When the light guide 41 may be possibly damaged in the middle, such as when a dark observation image is displayed on the monitor 7 or when no observation image is displayed thereon, the presence of a damage in the light guide is checked by examining an amount of illumination light emitted from the first to third light guides 41a to 41c. When it is determined that the light guide has damage, the light guide is replaced.

For the replacement, the threading 39 fixing the distal end of the casing tube 38 to the distal rigid part 31 in the endoscope distal end section 10 is first unthreaded, and the distal end of the casing tube 38 is rolled up to the rear part so that the inside of the endoscope distal end section 10 can be exposed.

Next, the fixing screw (not shown) fixing the image pickup unit 40 to the observation hole 131 of the distal rigid part 31 is unscrewed from the side of the distal rigid part 31, and the image pickup unit 40 is then moved to the proximal rear part (that is, the right direction in FIGS. 3 and 4) by using a tool, not shown. Then, the image pickup unit 40 is removed from the observation hole 131 and radiant concave 135.

Next, the LG fiber bundles 41 inserting through the frame bodies 42 in the illumination holes 132 to 134 bored to the distal rigid part 31 and the metal pipes 43 attached to the periphery are moved and pulled out one by one obliquely rearwards along the insertion axes LA2 of the illumination holes 132 to 134 by using a tool, not shown.

In this case, since the observation hole 131 and the first to third illumination holes 132 to 134 are communicated through the radiant concave 135 and since the image pickup unit 40 is removed from the observation hole 131 and radiant concave 135, the place from which the image pickup unit 40 is removed can be used as a pull-out space for moving the metal pipes 43 obliquely rearwards. Thus, the metal pipes 43 can be pulled out smoothly.

In this case, in order to remove the third light guide 41c attached into the third illumination hole 134 and the metal pipe 43 attached therearound, for example, the stroke L in the direction along the insertion axis LA1 of the observation hole 131, which is required for the removal, covers from the distal end of the metal pipe 43 to the radiant concave 135 as shown in FIG. 10, and the stroke H in the direction orthogonal to the insertion axis LA1 here is:
H=L·tan θd

Thus, as shown in FIG. 8, when distance Lo between the inner circumference exposing to the bottom of the radiant concave 135 of the third illumination hole 134 and the inner wall surface opposite to the observation hole 131 is wider than the stroke H (H<Lo), the third light guide 41c and the metal pipe 43 attached to the outer circumference can be removed from the third illumination hole 134 smoothly.

This is also applicable to the first and second light guides 41a and 41b attached to the other illumination holes 132 and 133 and the metal pipes 43 attached to the outer circumferences.

After the first to third light guides 41a to 41c and the metal pipes 43 attached to the outer circumferences are removed from the respective illumination holes 132 to 134, the LG fiber bundles are pulled out from the connector portion 4 side of the universal cord 3a of the endoscope 1.

Then, new first to third light guides 41a to 41c are fed from the connector portion 4 of the universal cord 3a of the endoscope 1 toward the distal end section 10, and the light guide 41 branching off to the first to third light guides 41a to 41c on the distal side are attached to the frame bodies 42 attached to the illumination holes 132 to 134. Notably, the steps of attachment is reverse of the steps of removal.

In this way, in the distal rigid part 31 of this embodiment, the insertion axes LA2 of the first to third illumination holes 132 to 134 in the distal rigid part 31 into which the first to third light guides 41a to 41c are placed tilt outward in the direction of insertion by the tilting angles θb to θd, respectively, in the range of 0° to 15° with respect to the insertion axis LA1 of the image pickup unit 40 of the observation hole 131.

Furthermore, as shown in FIGS. 6 to 8, the proximal side of the distal rigid part 31 has the radiant concave 135 enclosing the first to third illumination holes 132 to 134 about the observation hole 131, and the observation hole 131 and the first to third illumination holes 132 to 134 communicate with each other through the radiant concave 135.

Thus, in order to replace the first to third light guides 41a to 41c, the image pickup unit 40 is first removed, and the space from which the image pickup unit 40 is removed including the radiant concave 135 can be used as a removal space for the first to third light guides 41a to 41c and the metal pipes 43 attached to the outer circumferences. Therefore, the replacement of the first to third light guides 41a to 41c can be performed smoothly.

Therefore, the repairability and assemblability can be improved for replacing the first to third light guide 41a to 41c to be placed in the first to third illumination holes 132 to 134 of the distal rigid part 31.

Furthermore, having the same diameter as a conventional distal end section 10, the distal rigid part 31 can improve the repairability and assemblability for replacing the first to third light guides 41a to 41c without increasing the diameter of the endoscope distal end section 10.

Furthermore, the three first to third light guides 41a to 41c and three first to third illumination lenses 34a to 36a to be placed into the three first to third illumination holes 132 to 134 can allow balanced wider irradiation of a subject. As a result, objective lens 33a having a wider angular field of view can allow secure observation of the image of the widely irradiated subject.

Variation examples thereof will be described below.

According to this embodiment, the insertion axes LA2 of the first to third illumination holes 132 to 134 in the distal rigid part 31 into which the first to third light guides 41a to 41c are placed tilt outward in the direction of insertion by the tilting angles θb to θd, respectively, in the range of 0° to 15° with respect to the insertion axis LA1 of the image pickup unit 40 of the observation hole 131.

However, the present invention is not limited thereto, but the repairability and assemblability can be improved for only replacing a light guide placed in a tilting illumination hole if at least one insertion axis LA2 of the insertion axes LA2 of the first to third illumination holes 132 to 134 tilts outward in the direction of insertion by the tilting angles θb to θd, respectively, in the range of 0° to 15° with respect to the insertion axis LA1 of the image pickup unit 40 of the observation hole 131.

Furthermore, when the insertion axis LA2 of the first illumination hole 132 only tilts, the space between the distal opening 132s of the illumination hole 132 and the distal opening 131s of the observation hole 131 is more apart than the space between the proximal opening 132k of the illumination hole 132 and the proximal opening 131k of the observation hole 131.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. An endoscope distal end part comprising:
a first light guide placing through hole in which a first light guide is to be placed;
a second light guide placing through hole in which a second light guide is to be placed;
a third light guide placing through hole in which a third light guide is to be placed; and
an image pickup unit placing hole which is at least partially positioned in an area surrounded by each of the light guide placing through holes and in which an image pickup unit is to be placed.

2. The endoscope distal end part according to claim 1, further comprising:
a suction hole in an area between the first light guide placing through hole and the second light guide placing through hole;
an air/water feeding hole in an area between the second light guide placing through hole and the third light guide placing through hole; and
a front water feeding hole in an area between the third light guide placing through hole and the first light guide placing through hole.

3. The endoscope distal end part according to claim 1, wherein:
each of the light guide placing through holes and the image pickup unit placing hole on the proximal side communicate with each other through a concave.

4. The endoscope distal end part according to claim 1, wherein:
the distal and proximal ends of the light guide placing through holes are open on the distal side and proximal side; and
each opening of the light guide placing through holes on the distal side are spaced more apart than the spaces among the openings on the proximal side.

5. The endoscope distal end part according to claim 4, wherein:
the distal and proximal ends of the image pickup unit placing hole are open on the distal side and proximal side;
the center of the opening on the distal side of the first light guide placing through hole is on a first line connecting the center of the opening on the distal side of the first light guide placing through hole and the center of the opening on the distal side of the image pickup unit placing hole such that the angle formed by the first line and a second line connecting the center of the opening on the distal side of the second light guide placing through hole and the center of the opening on the distal side of the image pickup unit placing hole can be 90° to 150°;
the center of the opening on the distal side of the second light guide placing through hole is on the second line;
the center of the opening on the distal side of the third light guide placing through hole is on a third line connecting the center of the opening on the distal side of the third light guide placing through hole and the center of the opening on the distal side of the image pickup unit placing hole such that the angle formed by the third line and at least one of the first line and the second line can be 90° to 150°.

6. The endoscope distal end part according to claim 5, wherein:
the center of the opening on the distal side of the first light guide placing through hole is on the first line such that the angle formed by the first line and the second line can be 120°;
the center of the opening on the distal side of the second light guide placing through hole is on the second line; and
the center of the opening on the distal side of the third light guide placing through hole is on the third line such that the angle formed by the third line and at least one of the first line and the second line can be 120°.

7. The endoscope distal end part according to claim 1, further comprising:
a first insertion axis of the first light guide placing through hole for inserting the first light guide;
a second insertion axis of the second light guide placing through hole for inserting the second light guide;
a third insertion axis of the third light guide placing through hole for inserting the third light guide; and
an image pickup unit insertion axis of the image pickup unit placing hole for inserting the image pickup unit,
wherein at least one of the first insertion axis, the second insertion axis and the third insertion axis tilts with respect to the image pickup unit insertion axis.

8. The endoscope distal end part according to claim 7, wherein at least one of the first insertion axis, the second insertion axis and the third insertion axis tilts with respect to the image pickup unit insertion axis by 0° to 15°.

9. An endoscope distal end part comprising:
a first light guide placed in a first light guide placing through hole;
a second light guide placed in a second light guide placing through hole;
a third light guide placed in a third light guide placing through hole; and
an image pickup unit placed in an image pickup unit placing hole which is at least partially positioned in an area surrounded by each of the light guide placing through holes.

10. The endoscope distal end part according to claim 9, wherein each of the light guides is a light guide fiber bundle for guiding illumination light from a light source to irradiate to a subject.

11. The endoscope distal end part according to claim 10, wherein the image pickup unit includes a wide-angle observation optical system for observing the subject lighted by the illumination light and an image pickup device for picking up an image of the subject observed by the observation optical system.

12. The endoscope distal end part according to claim 9, further comprising:
a suction hole in an area between the first light guide and the second light guide;
an air/water feeding hole in an area between the second light guide and the third light guide; and
a front water feeding hole in an area between the third light guide and the first light guide.

13. The endoscope distal end part according to claim 9, wherein:
each of the light guide placing through holes and the image pickup unit placing hole on the proximal side communicate with each other through a concave.

14. The endoscope distal end part according to claim 13, wherein:
the distal and proximal ends of the light guide placing through holes are open on the distal side and proximal side; and
each opening of the light guide placing through holes on the distal side are spaced more apart than the spaces among the openings on the proximal side.

15. The endoscope distal end part according to claim 14, further comprising:
distal and proximal openings of the image pickup unit placing hole are open on the distal side and proximal side;
a first illumination lens which is placed in the distal opening of the first light guide placing through hole and is abutted on the distal end of the first light guide;
a second illumination lens which is placed in the distal opening of the second light guide placing through hole and is abutted on the distal end of the second light guide;
a third illumination lens which is placed in the distal opening of the third light guide placing through hole and is abutted on the distal end of the third light guide; and
an observation optical system which is placed in the distal opening of the image pickup unit placing hole and is positioned in front of the image pickup unit.

16. The endoscope distal end part according to claim 15, wherein the observation optical system has a wide angular field of view.

17. The endoscope distal end part according to claim 15, wherein:
the center of the optical axis of the first illumination lens is on a first line connecting the center of the optical axis of the first illumination lens and the center of the optical axis of the observation optical system such that the angle formed by the first line and a second line connecting the center of the optical axis of the second illumination lens and the center of the optical axis of the observation optical system can be 90° to 150°;
the center of optical axis of the second illumination lens is on the second line;
the center of the optical axis of the third illumination lens is on a third line connecting the center of optical axis of the third illumination lens and the center of the optical axis of the observation optical system such that the angle formed by the third line and at least one of the first line and the second line can be 90° to 150°.

18. The endoscope distal end part according to claim 17, wherein:
the center of the optical axis of the first illumination lens is on the first line such that the angle formed by the first line and the second line can be 120°;
the center of the optical axis of the second illumination lens is on the second line; and
the center of the optical axis of the third illumination lens is on the third line such that the angle formed by the third line and at least one of the first line and the second line can be 120°.

19. The endoscope distal end part according to claim 9, wherein at least one of the first light guide, the second light guide and the third light guide tilts with respect to the image pickup unit.

20. The endoscope distal end part according to claim 19, wherein at least one of the first light guide, the second light guide and the third light guide tilts with respect to the image pickup unit by 0° to 15°.

21. An endoscope distal end part comprising:
light guide placing through holes in which light guides are to be placed; and
an image pickup unit placing hole in which an image pickup unit is to be placed,
wherein:
the distal and proximal ends of the light guide placing through holes and image pickup unit placing hole are open on the distal side and proximal side; and
the openings of the light guide placing through holes on the distal side are spaced more apart than the spaces among the openings on the proximal side.

22. The endoscope distal end part according to claim 21, wherein:
the light guide placing through holes and the image pickup unit placing hole on the proximal side communicate with each other through a concave.

23. The endoscope distal end part according to claim 21, further comprising:
a light guide insertion axis of the light guide placing through hole for inserting the light guide; and
an image pickup unit insertion axis of the image pickup unit placing hole for inserting the image pickup unit,
wherein the light guide insertion axis tilts with respect to the image pickup unit insertion axis.

24. The endoscope distal end part according to claim 23, wherein the light guide insertion axis tilts with respect to the insertion axis of the image pickup unit by 0° to 15°.

25. The endoscope distal end part according to claim 21, wherein:
the light guide placing through holes in which the light guides are to be placed include three holes of a first light guide placing through hole, a second light guide placing through hole and a third light guide placing through hole; and
the image pickup unit placing hole is at least partially positioned in an area surrounded by each of the light guide placing through holes.

26. The endoscope distal end part according to claim 25, further comprising:
a suction hole in an area between the first light guide placing through hole and the second light guide placing through hole;
an air/water feeding hole in an area between the second light guide placing through hole and the third light guide placing through hole; and
a front water feeding hole in an area between the third light guide placing through hole and the first light guide placing through hole.

27. The endoscope distal end part according to claim 25, wherein:
the distal and proximal ends of each of the light guide placing through holes are open on the distal side and proximal side;
the center of the opening on the distal side of the first light guide placing through hole is on a first line connecting the center of the opening on the distal side of the first light guide placing through hole and the center of the opening on the distal side of the image pickup unit placing hole such that the angle formed by the first line and a second line connecting the center of the opening on the distal side of the second light guide placing through hole and the center of the opening on the distal side of the image pickup unit placing hole can be 90° to 150°;
the center of the opening on the distal side of the second light guide placing through hole is on the second line;
the center of the opening on the distal side of the third light guide placing through hole is on a third line connecting the center of the opening on the distal side of the third light guide placing through hole and the center of the opening on the distal side of the image pickup unit placing hole such that the angle formed by the third line and at least one of the first line and the second line can be 90° to 150°.

28. The endoscope distal end part according to claim 27, wherein:
the center of the opening on the distal side of the first light guide placing through hole is on the first line such that the angle formed by the first line and the second line can be 120°;
the center of the opening on the distal side of the second light guide placing through hole is on the second line; and
the center of the opening on the distal side of the third light guide placing through hole is on the third line such that the angle formed by the third line and at least one of the first line and the second line can be 120°.

29. An endoscope distal end part comprising:
light guides placed in light guide placing through holes; and
an image pickup unit placed in an image pickup unit placing hole,
wherein the distal and proximal ends of the light guide placing through holes are open on the distal side and proximal side; and
the space between the openings of the light guide placing through hole on the distal side and the opening on the distal side of the image pickup unit placing hole is more apart than the space between the openings of the light guide placing through hole on the proximal side and the opening on the proximal side of the image pickup unit placing hole.

30. The endoscope distal end part according to claim 29, wherein each of the light guides is a light guide fiber bundle for guiding illumination light from a light source to irradiate to a subject.

31. The endoscope distal end part according to claim 30, wherein the image pickup unit includes a wide-angle observation optical system for observing the subject lighted by the illumination light and an image pickup device for picking up an image of the subject observed by the observation optical system.

32. The endoscope distal end part according to claim 29, wherein:
the light guide placing through holes and the image pickup unit placing hole on the proximal side communicate with each other through a concave.

33. The endoscope distal end part according to claim 29, wherein the light guides tilt with respect to the image pickup unit.

34. The endoscope distal end part according to claim 33, wherein the light guides tilt with respect to the image pickup unit by 0° to 15°.

35. The endoscope distal end part according to claim 29, wherein:
the light guides placed in the light guide placing through holes include three of a first light guide, a second light guide and a third light guide;
the image pickup unit is at least partially positioned in an area surrounded by the light guides.

36. The endoscope distal end part according to claim 35, further comprising:
a suction hole in an area between the first light guide and the second light guide;
an air/water feeding hole in an area between the second light guide and the third light guide; and
a front water feeding hole in an area between the third light guide and the first light guide.

37. The endoscope distal end part according to claim 35, further comprising:
distal and proximal openings of each of the light guide placing through holes and image pickup unit placing hole on the distal side and proximal side;
a first illumination lens which is placed in the distal opening of the first light guide placing through hole and is abutted on the distal end of the first light guide;
a second illumination lens which is placed in the distal opening of the second light guide placing through hole and is abutted on the distal end of the second light guide;
a third illumination lens which is placed in the distal opening of the third light guide placing through hole and is abutted on the distal end of the third light guide; and
an observation optical system which is placed in the distal opening of the image pickup unit placing hole and is positioned in front of the image pickup unit.

38. The endoscope distal end part according to claim 37, wherein the observation optical system has a wide angular field of view.

39. The endoscope distal end part according to claim 37, wherein:
the center of the optical axis of the first illumination lens is on a first line connecting the center of the optical axis of the first illumination lens and the center of the optical axis of the observation optical system such that the angle formed by the first line and a second line connecting the center of the optical axis of the second illumination lens and the center of the optical axis of the observation optical system can be 90° to 150°;
the center of optical axis of the second illumination lens is on the second line;
the center of the optical axis of the third illumination lens is on a third line connecting the center of optical axis of the third illumination lens and the center of the optical axis of the observation optical system such that the angle formed by the third line and at least one of the first line and the second line can be 90° to 150°.

40. The endoscope distal end part according to claim 39, wherein:
the center of the optical axis of the first illumination lens is on the first line such that the angle formed by the first line and the second line can be 120°;
the center of the optical axis of the second illumination lens is on the second line; and
the center of the optical axis of the third illumination lens is on the third line such that the angle formed by the third line and at least one of the first line and the second line can be 120°.

41. An endoscope distal end part comprising:
first light guide placing means in which a first light guide is to be placed;
second light guide placing means in which a second light guide is to be placed;
third light guide placing means in which a third light guide is to be placed; and
image pickup unit placing means which is at least partially positioned in an area surrounded by each of the light guide placing means and in which an image pickup unit is to be placed.

42. The endoscope distal end part according to claim 41, wherein the light guide placing means and the image pickup unit placing means are holes having open distal and proximal ends on the distal and proximal sides.

43. The endoscope distal end part comprising:
first illumination means placed in a first light guide placing through hole;
second illumination means placed in a second light guide placing through hole;
third illumination means placed in a third light guide placing through hole; and
image pickup means placed in an image pickup unit placing hole, which is at least partially positioned in an area surrounded by each of the illumination means.

44. The endoscope distal end part according to claim 43, wherein each of the illumination means is a light guide fiber bundle for guiding illumination light from a light source to irradiate to a subject.

45. The endoscope distal end part according to claim 43, wherein the image pickup means is an image pickup unit including a wide-angle observation optical system for observing the subject lighted by the illumination light and an image pickup device for picking up an image of the subject observed by the observation optical system.

46. An endoscope distal end part comprising:
light guide placing means in which light guides are to be placed; and
image pickup unit placing means in which an image pickup unit is to be placed,
wherein:
the distal and proximal ends of the light guide placing means and image pickup unit placing means are open on the distal side and proximal side; and
the space between the openings of the light guide placing means on the distal side and the opening on the distal side of the image pickup unit placing means is more apart than the space between the openings of the light guide placing means on the proximal side and the opening on the proximal side of the image pickup unit placing means.

47. The endoscope distal end part according to claim 46, wherein:
each of the light guide placing means is a hole having the distal and proximal openings, which communicate with each other; and
the image pickup unit placing means is a different hole from the hole for the light guide placing means.

48. An endoscope distal end part comprising:
illumination means placed in light guide placing through hole; and
image pickup means placed in an image pickup unit placing hole,
wherein:
the distal and proximal ends of the light guide placing through hole and image pickup unit placing hole are open on the distal side and proximal side; and
the space between the openings of the light guide placing through hole on the distal side and the opening on the distal side of the image pickup unit placing hole is more apart than the space between the openings of the light guide placing through hole on the proximal side and the opening on the proximal side of the image pickup unit placing hole.

49. The endoscope distal end part according to claim 48, wherein illumination means is a light guide fiber bundle for guiding illumination light from a light source to irradiate to a subject.

50. The endoscope distal end part according to claim 49, wherein the image pickup means is an image pickup unit including a wide-angle observation optical system for observing the subject lighted by the illumination light and an image pickup device for picking up an image of the subject observed by the observation optical system.
